(19) United States
(12) Patent Application Publication
(10) Pub. No.: US 2006/0154373 A1
(43) Pub. Date: Jul. 13, 2006

(54) Easy scan fecal blood test

(76) Inventor: William Thomas Buchanan, Murchison, TX (US)

Correspondence Address: William T. Buchanan, 8213 East Lake, Murchison, TX, 75778, US

(21) Appl. No.: 11/032,479

(22) Filed: Jan. 11, 2005

(51) Int. Cl. G01N 33/48

(52) U.S. Cl. 436/66

(57) ABSTRACT

Fecal Blood Tests are the primary screening methods for finding blood in the digestive system and indications for colorectal diseases. Almost all of the current tests are performed with the supervision of physicians, and are quite messy, and expensive. Consequently, the tests are not utilized and cause the deaths of thousands, plus 58,000 estimated in the United States in 2005, that could be prevented. The Easy Scan Fecal Blood Test is an easy-to-use, clean, home, fecal blood test made available to thousands of individuals by including the test with the purchase of toilet tissue at essentially no cost.

### CROSS REFERENCE TO RELATED APPLICATIONS

| No. | Date | Inventor |
|---|---|---|
| 5,954,369 | Sep. 21, 1999 | Seabrook, March E. |

### BACKGROUND OF THE INVENTION

In 2005 it is estimated there will be almost 58,000 deaths in the United States alone, due to colorectal diseases, such as cancer, ulcers, colitis, etc. Most of these deaths can be prevented with early detection, but too few people get regular screening for these diseases.

A fecal blood test is one screening method but most of these tests normally are originated by physicians and are quite unsanitary and messy. Home detection kits are available but again are not widely used.

The Easy Screen Fecal Blood Test is easy-to-use and clean, and including this test coupled with toilet tissue will cause thousands of early detections of these insidious diseases.

### BRIEF SUMMARY OF THE INVENTION

The Easy Screen Fecal Blood Test is an easy-to-use, clean, home fecal blood-screening test that is coupled with the marketing of toilet tissue.

### DETAILED DESCRIPTION

The Easy Screen Fecal Blood Test is an easy-to-use, clean, home fecal blood-screening test that is coupled with the general marketing and sales of toilet tissue. The test consists of a sealed packet of treated paper, which turns a blue-green color when contaminated with blood.

The Easy Screen Fecal Blood Test is easy-to-use and clean. Simply remove all toilet cleaners, etc. from the bowl and the tank. Flush several times. After a bowel movement, remove the test patch and float it in the water with the stool. A blue-green color development within three minutes is an indication of blood in the stool and a possible medical problem. Contact your physician for further testing.

In compliance with the statute, the invention has been described in language more or less specific as to the structural features. It is understood, however, that the invention is not limited to the specific features described, since the means and construction herein disclosed comprise a preferred form of putting the invention into effect. The invention is, therefore, claimed in any of its forms or modifications within the proper scope of the appended claims interpreted in accordance with the doctrine of equivalents.

1. I claim a system for providing a free or low cost, easy-to-use, clean, home, fecal blood-screening test, to individuals comprising of a separate test paper or card to be included with the packaging and general marketing of toilet tissue, either with a single roll or multiple rolls. The test is utilized by simply dropping the test paper or card in toilet water containing fecal matter and requires no handling, packaging or shipping of the test paper or card to a physician.
